(19) United States
(12) Patent Application Publication
(10) Pub. No.: US 2026/0363980 A1
(43) Pub. Date: Nov. 19, 2026

(54) GENTLE CLEANSING COMPOSITION

(71) Applicant: L'OREAL, Paris (FR)

(72) Inventors: Xuefei LIU, Edison, NJ (US); Hy Si BUI, Piscataway, NJ (US); Ritesh Kumar SINHA, Linden, NJ (US)

(73) Assignee: L'OREAL, Paris (FR)

(21) Appl. No.: 19/207,541

(22) Filed: May 14, 2025

(51) Int. Cl. A61K 8/37; A61K 8/06; A61K 8/34; A61K 8/63; A61K 8/73; A61Q 19/08; A61Q 19/10

(52) U.S. Cl. CPC A61K 8/37; A61K 8/062; A61K 8/342; A61K 8/63; A61K 8/735; A61Q 19/08; A61Q 19/10

(57) ABSTRACT

A gentle cleansing composition comprising: (a) brassicyl valinate esylate; (b) one or more fatty alcohols; (c) one or more nonionic surfactants; (d) one or more skin active agents; (e) one or more water soluble solvents; and (f) water is disclosed. The composition comprises multilamellar vesicles, wherein the brassicyl valinate esylate is entrapped within the vesicles. Also disclosed are methods for cleansing the skin and face with the gentle cleansing composition.

### FIELD OF THE DISCLOSURE

The present disclosure relates to gentle cleansing compositions that are particular well-suited for the face and to methods for gently cleansing skin, including facial skin.

### BACKGROUND

Surfactants are widely used in aqueous based personal care, household, and industrial products. They are typically used as wetting agents, detergents, and emulsifiers. In personal care cleansing products (e.g., shampoos, body washes, facial cleansers, liquid hand soaps, etc.) the surfactant is often the most important component because it provides many of the cleansing attributes of the composition.

Although in principle any surfactant class (e.g., cationic, anionic, nonionic, amphoteric) is suitable in cleansing or cleaning applications, in practice most personal care cleansers and household cleaning products are formulated with anionic surfactants or with a combination of an anionic surfactant as the primary detersive agent with one or more secondary surfactants selected from the other surfactant classes. Anionic surfactants are often used as detersive agents in cleansers and cleaning products because of their excellent cleaning and foaming properties. From the consumer's perspective, the amount and stability of the foam directly relates to the perceived cleaning efficiency of the composition. Generally speaking, the larger the volume of foam produced and the more stable the foam, the more efficient is the perceived cleaning action of the composition. This presents a potential problem in low-surfactant formulations, as foam volume tends to decrease with decreasing surfactant concentration.

Sulfate-based surfactants (such as, for example, sodium lauryl sulfate and sodium lauryl ether sulfate) are particularly popular because of their effectiveness in cleansing, foam production, and stability. Personal care cleansers containing sulfate-based surfactants are also generally easy to thicken with typical thickeners, such as salt and cellulose-based materials. Nonetheless, these particular surfactants can be harsh and irritating to skin. For instance, over-use of sulfate-based surfactants can cause needless drying to the face and scalp, and contribute to color fading and drying of hair. Eliminating sulfate surfactants from cleansing compositions has been challenging because sulfate-free compositions typically have poor foaming properties, are difficult to thicken, are not clear (not transparent).

Consumers increasingly seek gentle cleansing compositions that effectively remove dirt, oil, and makeup without stripping the skin of its natural moisture. Products that offer hydration and moisture retention are needed to help maintain optimal skin hydration levels.

### SUMMARY OF THE DISCLOSURE

The gentle cleansing compositions of the instant disclosure leverage an advanced delivery system, with multilamellar vesicles, to allow for a controlled release of hydration throughout the day. The compositions effectively remove dirt, oil, and makeup but, unlike typical cleansing compositiond, they promote optimal skin hydration. In addition, the gentle cleansing compositions deliver skin active agents that support the skin's natural barrier, ensuring long-term health and protection. These gentle cleansing composition typically include:
(a) brassicyl valinate esylate;
(b) one or more fatty alcohols;
(c) one or more nonionic surfactants;
(d) one or more skin active agents; and
(e) one or more water soluble solvents;
(f) water;
   wherein the composition contains multilamellar vesicles, and the brassicyl valinate esylate is entrapped within the vesicles.

Multilamellar vesicles (MLVs) represent a liposomal delivery system characterized by their unique concentric layered structure, resembling an onion. These structures are formed by multiple lipid bilayers, each separated by an aqueous compartment, allowing for the encapsulation of both hydrophilic and lipophilic active ingredients within different regions of the MLV structure. Hydrophilic actives can be incorporated within the aqueous layers, while lipophilic actives are embedded within the lipid bilayers themselves, enabling efficient co-delivery of actives with different solubility profiles (Giuliano, Camila Betterelli, et al. “Multivesicular vesicles: preparation and applications.” ChemSystemsChem 3.2 (2021): e2000049, which is incorporated herein by reference in its entirety).

The formation of stable and well-defined MLVs can be further enhanced by the judicious selection and combination of specific components, including fatty alcohols and surfactants with tailored properties. Fatty alcohols, such as cetyl alcohol, stearyl alcohol, or behenyl alcohol, can be incorporated into the lipid bilayers, influencing their physical characteristics and stability. These long-chain alcohols increase the rigidity and order of the lipid bilayers, thereby modulating the permeability and release kinetics of encapsulated actives. Moreover, the presence of fatty alcohols can improve the overall stability of the MLVs, preventing aggregation or fusion of the vesicles (Iwata, T. “Lamellar gel network.” Cosmetic Science and Technology: Theoretical Principles and Applications (2017): 415-447, which is incorporated herein by reference in its entirety).

The size of MLVs typically ranges from a few hundred nanometers to several micrometers in diameter. Their multilayered structure is clearly distinguishable under polarized light microscopy, where they exhibit a characteristic spherical “Maltese cross” birefringence pattern. This optical property arises from the interaction of polarized light with the ordered arrangement of lipid molecules within the concentric bilayers, a feature that differentiates them from sheet-like lamellar structures (Marques, Eduardo F., and Ali Khan. “Effect of a bile salt on the aggregation behavior of a double-chained cationic surfactant—the cationic-rich dilute region of the didodecyldimethylammonium bromide-sodium taurodeoxycholate-water system.” Lipid and Polymer-Lipid Systems. Springer Berlin Heidelberg, 2002, which is incorporated herein by reference in its entirety).

It has been reported that MLVs designed for topical applications can provide controlled and sustained release of encapsulated actives. The multiple bilayers function as diffusion barriers, significantly slowing the release of the ingredients compared to conventional delivery systems. This controlled release profile offers several advantages, including enhanced efficacy, reduced irritation, and improved formulation stability (Agnihotri, Sunil A., Kumaresh S. Soppimath, and Guru V. Betageri. “Controlled release application of multilamellar vesicles: a novel drug delivery approach.” Drug Delivery 17.2 (2010): 92-101, which is incorporated herein by reference in its entirety).

In addition, encapsulation efficiencies of various actives-such as steroids-within MLV liposomes have been well-documented, underscoring the flexibility of MLVs as delivery vehicles (Kulkarni, S. B., and E. I. Vargha-Butler. “Study of liposomal drug delivery systems 2. Encapsulation efficiencies of some steroids in MLV liposomes.” Colloids and Surfaces B: Biointerfaces 4.2 (1995): 77-85, which is incorporated herein by reference in its entirety).

Nonlimiting examples of nonionic surfactants include those selected from alkyl polyglucosides; alcohols, alpha-diols, alkylphenols and esters of fatty acids, being ethoxylated, propoxylated or glycerolated (polyglyceryl-2 isostearate); ethoxylated fatty esters; glyceryl esters of fatty acids; fatty alcohol ethoxylates; alkyl phenol ethoxylates; fatty acid alkoxylates; and mixtures thereof. Preferably, at least one of the one or more nonionic surfactants is an alkoxylated nonionic surfactant. Alkoxylated nonionic surfactants are a class of surfactants formed by the reaction of hydrophilic molecules, such as alcohols, phenols, or fatty acids, with alkylene oxides like ethylene oxide (EO) or propylene oxide (PO). Nonlimiting examples include alkoxylated polysorbates, alkoxylated sorbitan esters, alkoxylated fatty ethers, alkoxylated fatty acids, alkoxylated glyceryl esters, or combinations thereof. In various embodiments, the alkoxylated nonionic surfactants are selected from ethoxylated fatty alcohols, polysorbates, PEG derivatives, and poloxamers.

Nonlimiting examples of fatty alcohols include those having at least 8 carbon atoms and are linear or branched. In various embodiments, the one or more fatty alcohols have from 10 to 30 carbon atoms, preferably from 12 to 28 carbon atoms, for example, capryl alcohol, pelargonic alcohol, decyl alcohol, undecyl alcohol, lauryl alcohol, tridecyl alcohol, myristyl alcohol, pentadecyl alcohol, cetyl alcohol, palmitoleyl alcohol, isostearyl alcohol, isocetyl alcohol, heptadecyl alcohol, stearyl alcohol, cetearyl alcohol, oleyl alcohol, nonadecyl alcohol, arachidyl alcohol, heneicosyl alcohol, behenyl alcohol, erucyl alcohol, lignoceryl alcohol, ceryl alcohol, 1-heptacosanol, montanyl alcohol, 1-nonacosanol, and myricyl alcohol.

The one or more fatty alcohols may include liquid fatty alcohols, solid fatty alcohols, or combinations thereof. Nonlimiting examples of solid fatty alcohol include stearyl alcohol, cetyl alcohol, cetearyl alcohol alcohol, myristyl alcohol, behenyl alcohol, arachidyl alcohol, and mixtures thereof. Nonlimiting examples of liquid fatty alcohols include isostearyl alcohol, oleyl alcohol, octyldodecanol, linoleyl alcohol, linolenyl alcohol, lauryl alcohol, 2-hexyldecanol, 2-heptyldecanol, enanthic alcohol (1-heptanol), caprylic alcohol (1-octanol), pelargonic alcohol (1-nonanol), capric alcohol (1-decanol), or mixtures thereof. In a preferred embodiment, the one or more liquid fatty alcohols are selected from lauryl alcohol, oleyl alcohol, octyldodecanol, and mixtures thereof.

Nonlimiting examples of water soluble solvents include C₂-C₆ monoalcohols, polyols (polyhydric alcohols), glycerin, glycols, or a combination thereof. The polyols preferably have two or three hydroxyl groups. Nonlimiting examples of include ethylene glycol, propylene glycol, butylene glycol, hexylene glycol, pentylene glycol, 1,3-propanediol, diethylene glycol, dipropylene glycol, caprylyl glycol, glycerin, and a combination thereof.

Nonlimiting examples (categories) of skin active agents include antioxidants, anti-wrinkle agents, depigmenting agents, vitamins, ceramides, alpha and/or beta hydroxy acids, anti-acne agents, etc. More specific but nonlimiting examples of skin active agents include vitamin A, vitamin C, vitamin D, vitamin E), niacinamide, caffeine, ceramides, ferulic acid, salicylic acid, madecassoside, retinoic acid, benzoyl peroxide, or a combination thereof.

The gentle cleansing composition optionally includes one or more thickening polymers. Nonlimiting examples of thickening polymers include polyacrylate polymers, polyacrylamide polymers, polysaccharides, gums (e.g., guar gums), carbomers, and a combination thereof.

The gentle cleansing composition is particularly useful for cleansing the body, especially the skin, including the skin of the face. When cleansing and/or removing makeup from the skin, the compositions may be applied to the skin and rinsed from the skin with water. As mentioned above, the cleansing compositions are gentle to the skin and are unique in their ability to hydrate the skin. Therefore, the compositions may be used in methods for gently cleansing and hydrating the skin.

### DETAILED DESCRIPTION OF THE DISCLOSURE

The cleansing composition of the present disclosure typically include:
(a) about 0.1 to about 5 wt. % of brassicyl valinate esylate;
(b) about 0.5 to about 5 wt. % of one or more fatty alcohols;
(c) about 1 to about 8 wt. % of one or more nonionic surfactants;
(d) about 0.01 to about 5 wt. % of one or more skin active agents; and
(e) about 1 to about 15 wt. % of one or more water soluble solvents;
(f) water;
   wherein the composition contains multilamellar vesicles, wherein the brassicyl valinate esylate is entrapped within the vesicles, and all weight percentages are based on a total weight of the composition.

### (a) Brassicyl Valinate Esylate

Brassicyl valinate esylate is typically derived from plant-based sources, specifically from Brassica (such as rapeseed or mustard). It contains valine (an amino acid) and at lower pH values can become protonated.

The amount of the brassicyl valinate eslyate in the gentle cleansing composition ill vary but is typically from about 0.1 to about 5 wt. %, based on the total weight of the gentle cleansing composition. In further embodiments, the gentle cleansing composition includes from about 0.1 to about 4 wt. %, about 0.1 to about 3 wt. %, about 0.1 to about 2 wt. %, about 0.1 to about 1 wt. %, about 0.2 to about 5 wt. %, 0.2 to about 4 wt. %, about 0.2 to about 3 wt. %, about 0.2 to about 2 wt. %, about 0.2 to about 1 wt. %, about 0.3 to about 5 wt. %, 0.3 to about 4 wt. %, about 0.3 to about 3 wt. %, about 0.3 to about 2 wt. %, about 0.3 to about 1 wt. %, about 0.4 to about 5 wt. %, 0.4 to about 4 wt. %, about 0.4 to about 3 wt. %, about 0.4 to about 2 wt. %, about 0.4 to about 1 wt. %, or about 0.4 to about 0.8 wt. % of the brassicyl valinate eslyate, based on the total weight of the gentle cleansing composition.

In a preferred embodiment, the gentle cleansing composition includes from about 0.1 to about 4 wt. %, more preferably about 0.2 to about 3 wt. %, even more preferably about 0.3 to about 2 wt. % of the brassicyl valinate eslyate, based on the total weight of the gentle cleansing composition.

### (b) Fatty Alcohols

The term “fatty alcohol” means an alcohol comprising at least one hydroxyl group (OH), and typically comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In various embodiments, the hair treatment composition includes at least one solid fatty alcohol. Solid fatty alcohols are fatty alcohols that are solid at ambient temperature and at atmospheric pressure (25 °C, 780 mmHg), and are insoluble in water, i.e., they have a water solubility of less than 1% by weight, preferably less than 0.5% by weight, at 25 °C, 1 atm. The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms. Nonlimiting examples include lauryl alcohol (1-dodecanol); myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricylic alcohol (1-triacontanol), and combinations thereof. In a preferred embodiment, the compositions include at least one solid fatty alcohol selected from myristyl alcohol, cetyl alcohol, stearyl alcohol, behenyl alcohol and combinations thereof such as cetylstearyl or cetearyl alcohol.

In various embodiments, the hair treatment composition includes at least one liquid fatty alcohol, in particular containing C10-C34 and preferably have branched carbon chains and/or have one or more, preferably 1 to 3 double bonds. They are preferably branched and/or unsaturated (C═C double bond) and contain from 12 to 40 carbon atoms. The liquid fatty alcohols may be represented by: R—OH, wherein R denotes a C12-C24 branched or straight alkyl group or an alkenyl group, R being optionally substituted by one or more hydroxy groups. In certain embodiments, the liquid fatty alcohols are selected from branched saturated alcohols. Preferably, R does not contain a hydroxyl group. Nonlimiting examples include oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and combinations thereof. In other embodiments, the compositions are free or essentially free from liquid fatty alcohols, including the liquid fatty alcohols referenced above.

In a preferred embodiment, the one or more fatty alcohols are linear (straight chain) saturated fatty alcohols with 10 to 30 carbon atoms, preferably from 12 to 28 carbon atoms, more preferably from 14 to 24 carbon atoms. Nonlimiting examples include decyl alcohol, undecyl alcohol, dodecyl alcohol, myristyl alcohol, cetyl alcohol, stearyl alcohol, cetearyl alcohol, behenyl alcohol, myricyl alcohol and combinations thereof.

The total amount of the one or more fatty alcohols in the gentle cleansing composition will vary but is typically from about 0.1 to about 10 wt. %, based on the total weight of the gentle cleansing composition. In further embodiments, the gentle cleansing composition includes from about 0.1 to about 8 wt. %, about 0.1 to about 6 wt. %, about 0.1 to about 5 wt. %, about 0.2 to about 10 wt. %, about 0.2 to about 8 wt. %, about 0.2 to about 6 wt. %, about 0.2 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, or about 1 to about 4 wt. %, based on the total weight of the gentle cleansing composition.

Preferably, the gentle cleansing composition includes from about 0.5 to about 8 wt. %, more preferably about 0.8 to about 6 wt. %, and even more preferably about 1 to about 5 wt. % of one or more fatty alcohols, based on the total weight of the gentle cleansing composition.

As noted above, fatty alcohol include solid fatty alcohol and liquid fatty alcohols. Nonlimiting examples of both are provided below.

### Solid Fatty Alcohols

The term “solid fatty alcohol” means a fatty alcohol having a melting point greater than 27 °C at atmospheric pressure (1.013×10⁵ Pa). For the purposes of the present disclosure, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments.

The term “fatty alcohol” means a long-chain aliphatic alcohol comprising from 8 to 40 carbon atoms, preferably from 12 to 34 or even from 12 to 30 carbon atoms and comprising at least one hydroxyl group OH. These fatty alcohols are neither oxyalkylenated nor glycerolated. Preferably, the solid fatty alcohols are of structure R—OH with R denoting a linear alkyl group, optionally substituted with one or more hydroxyl groups, comprising from 12 to 40, better still from 12 to 34, or even from 12 to 30, and most preferentially from 12 to 24 carbon atoms.

Nonlimiting examples of solid fatty alcohols include myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricyl alcohol (1-triacontanol), and mixtures thereof. In a preferred embodiment, the one or more solid fatty alcohols are selected from myristyl alcohol, cetyl alcohol, stearyl alcohol, behenyl alcohol and mixtures thereof, such as cetylstearyl alcohol/cetearyl alcohol.

The total amount of the one or more solid fatty alcohols in the gentle cleansing compositions will vary, but is typically from 0.1 to about 8 wt. %, based on the total weight of the gentle cleansing composition. In further embodiments, the gentle cleansing composition includes from about 0.1 to about 6 wt. %, about 0.1 to about 5 wt. %, about 0.1 to about 4 wt. %, about 0.1 to about 3 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.8 to about 8 wt. %, about 0.8 to about 6 wt. %, about 0.8 to about 5 wt. %, about 0.8 to about 4 wt. %, about 0.8 to about 3 wt. %, about 1 to about 8 wt. %, about 1 to about 6 wt. %, about 1 to about 5 wt. %, about 1 to about 4 wt. %, about 1 to about 3 wt. %, or about 1.2 to about 3 wt. % of one or more liquid fatty alcohols, based on the total weight of the gentle cleansing composition.

Preferably, the gentle cleansing composition includes from about 0.5 to about 5 wt. %, more preferably about 1 to about 4 wt. %, and even more preferably about 1.5 to about 3 wt. % of one or more solid fatty alcohol, based on the total weight of the gentle cleansing composition.

### Liquid Fatty Alcohols

The term “liquid fatty alcohol” means a fatty alcohol having a melting point of less than or equal to 27 °C at atmospheric pressure (1.013×10⁵ Pa). For the purposes of the present disclosure, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments.

The liquid fatty alcohols may be linear or branched, saturated or unsaturated alcohols, preferably unsaturated or branched alcohols, including from 8 to 40 carbon atoms and preferably from 8 to 30 carbon atoms. These fatty alcohols are neither oxyalkylenated nor glycerolated. Nonlimiting examples include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol, linoleyl alcohol, isostearyl alcohol, octyldodecanol, lauryl alcohol, 2-hexyldecanol, 2-heptyldecanol, caproic alcohol (1-hexanol), enanthic alcohol (1-heptanol), caprylic alcohol (1-octanol), pelargonic alcohol (1-nonanol), capric alcohol (1-decanol), or mixtures thereof. In a preferred embodiment, the one or more liquid fatty alcohols are selected from octyldodecanol, isostearyl alcohol, lauryl alcohol, oleyl alcohol, linolenyl alcohol, ricinoleyl alcohol, undecylenyl alcohol, linoleyl alcohol, and mixtures thereof.

The total amount of the one or more liquid fatty alcohols in the gentle cleansing compositions will vary, but is typically from 0.1 to about 8 wt. %, based on the total weight of the gentle cleansing composition. In further embodiments, the gentle cleansing composition includes from about 0.1 to about 6 wt. %, about 0.1 to about 5 wt. %, about 0.1 to about 4 wt. %, about 0.1 to about 3 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.8 to about 8 wt. %, about 0.8 to about 6 wt. %, about 0.8 to about 5 wt. %, about 0.8 to about 4 wt. %, about 0.8 to about 3 wt. %, about 1 to about 8 wt. %, about 1 to about 6 wt. %, about 1 to about 5 wt. %, about 1 to about 4 wt. %, about 1 to about 3 wt. %, or about 1.2 to about 3 wt. % of the one or more liquid fatty alcohols, based on the total weight of the gentle cleansing composition.

Preferably, the gentle cleansing composition includes from about 0.5 to about 5 wt. %, more preferably about 1 to about 4 wt. %, and even more preferably about 1.5 to about 3 wt. % of one or more liquid fatty alcohol, based on the total weight of the gentle cleansing composition.

### Combined Amounts of (a) and (b)

The combined amount of the brassicyl valinate esylate of (a) and the one or more nonionic surfactants of (b) in the gentle cleansing composition will vary, but is typically from about 1.5 to about 10 wt. %, based on the total weight of the hair treatment composition. In further embodiments, the combined amount of (a) and (b) in the hair treatment composition is from about 1.5 to about 8 wt. %, about 1.5 to about 6 wt. %, about 1.5 to about 5 wt. %, about 1.5 to about 4 wt. %, about 2 to about 8 wt. %, about 2 to about 6 wt. %, about 2 to about 5 wt. %, about 2 to about 4 wt. %, about 2.5 to about 8 wt. %, about 2.5 to about 6 wt. %, about 2.5 to about 5 wt. %, about 2.5 to about 4 wt. %, about 3 toa bout 8 wt. %, about 3 to about 6 wt. %, about 3 to about 5 wt. %, or about 3 to about 4 wt. %, based on the total weight of the gentle cleansing composition.

Preferably, the combined amount of (a) and (b) is from about 1.5 to about 8 wt. %, more preferably about 2 to about 6 wt. %, even more preferably about 2.5 to about 5 wt. %, and even more preferably about 3 to about 4 wt. %, based on the total weight of the gentle cleansing composition.

The combined amount of the brassicyl valinate esylate of (a) and the one or more nonionic surfactants of (b) in the gentle cleansing composition will vary, but is typically from about 2 to about 12 wt. %, based on the total weight of the gentle composition. In further embodiments, the combined amount of (a) and (b) is from about 2 to about 10 wt. %, about 2 to about 8 wt. %, about 2 to about 6 wt. %, about 2 to about 5 wt. %, about 2 to about 4 wt. %, about 2.5 to about 12 wt. %, about 2.5 to about 10 wt. %, about 2.5 to about 8 wt. %, about 2.5 to about 6 wt. %, about 2.5 to about 5 wt. %, about 2.5 to about 4 wt. %, about 3 to about 12 wt. %, about 3 to about 10 wt. %, about 3 to about 8 wt. %, about 3 to about 6 wt. %, about 3 to about 5 wt. %, or about 5 to about 4 wt. %, based on the total weight of the gentle cleansing composition.

### (c) Nonionic Surfactants

A nonionic surfactant is a type of surfactant that does not carry an electrical charge in solution. Unlike anionic or cationic surfactants, which can have negatively or positively charged head groups, nonionic surfactants have a hydrophilic (water-attracting) head group that is uncharged, typically consisting of ethylene oxide or other polar functional groups.

Nonlimiting examples of nonionic surfactants include: alkanolamides; alkyl polyglucosides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; and combinations thereof.

In a preferred embodiment, one or more of the nonionic surfactants are selected from alkoxylated nonionic surfactants.

(c)(i) Alkanolamide Surfactants

Nonlimiting examples alkanolamide surfactants include fatty acid alkanolamide surfactants. The fatty acid alkanolamide surfactants may be fatty acid monoalkanolamides, fatty acid dialkanolamides, or fatty acid isoalkanolamides, and may have a C₂₋₈ hydroxyalkyl group (the C₂₋₈ chain can be substituted with one or more than one-OH group). Nonlimiting examples include fatty acid diethanolamides (DEA) or fatty acid monoethanolamides (MEA), fatty acid monoisopropanolamides (MIPA), fatty acid diisopropanolamides (DIPA), and fatty acid glucamides (glucamide surfactants).

Suitable fatty acid alkanolamides include those formed by reacting an alkanolamine and a C₆-C₃₆ fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and combinations thereof.

In some instances, the fatty acid alkanolamides preferably include cocamide MIPA, cocamide DEA, cocamide MEA, cocamide DIPA, and combinations thereof. In particular, the fatty acid alkanolamide may be cocamide MIPA, which is commercially available under the tradename “EMPILAN” from Innospec Active Chemicals.

Fatty acid alkanolamides surfactants include those of the following structure (XIV):

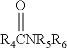

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and combinations thereof);
R₆ is selected from —CH₂OH, —CH₂CH₂OH, —CH₂CH₂CH₂OH, —CH₂(CHOH)₄CH₂OH, -benzyl, and combinations thereof;
R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and combinations thereof.

In some instances, the one or more of the fatty acid alkanolamides include one or more glucamide surfactants, for example, glucamides surfactants having a carbon chain length of 8 to 20. Nonlimiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide. Preferred glucamide surfactants include lauroyl/myristoyl methyl glucamide, cocamide methyl glucamide, lauramide methyl glucamide, myristamide methyl glucamide, caprylic/capric glucamide, oleamide methyl glucamide, stearamide methyl glucamide, behenamide methyl glucamide, palmitamide methyl glucamide, and combinations thereof.

Alkoxylated alkanolamide surfactants are a class of surfactants that combine the properties of alkanolamides with alkoxylation. Alkoxylation involves the addition of alkylene oxides (such as ethylene oxide or propylene oxide) to a chemical structure, which modifies its properties and enhances its functionality in various applications. Nonlimiting examples of alkoxylated alkanolamide surfactants include ethoxylated alkanolamides, for example, a rapeseedamide ethoxylate, a cocamide DEA ethoxylate, a cocamide MEA ethoxylate, a lauramide DEA ethoxylate, an oleamide DEA ethoxylate, a caprylic/capric acid diethanolamide ethoxylate, or combinations thereof. Preferably, the ethoxylated alkanolamide has from 2 to 100 ethoxylate groups. Further, the ethoxylated alkanolamide may have 2 to 80, 2 to 60, 2 to 50, 4 to 100, 4 to 80, 4 to 60, 4 to 50, 10 to 100, 10 to 80, 10 to 60, or 10 to 50 ethoxylate groups. Preferably, the alkanolamide is ethoxylated with 2 to 100 ethoxylate groups, more preferably with 2 to 60 ethoxylate groups, even more preferably with 2 to 40 ethoxylate groups, and even more preferably with 4 to 20 ethoxylate groups.

In a preferred embodiment, the cleansing composition includes a rapeseedamide ethoxylate, for example, one or more rapeseedamide ethoxylates selected from PEG-2 rapeseedamide, PEG-4 rapeseedamide, PEG-6 rapeseedamide, PEG-10 rapeseedamide, PEG-20 rapeseedamide, PEG-30 rapeseedamide, PEG-40 rapeseedamide, PEG-60 rapeseedamide, PEG-80 rapeseedamide.

(c)(ii) Polyglucoside Surfactants

Polyglucoside surfactants are primarily composed of glucose units linked together by glycosidic bonds. These bonds connect glucose molecules to form a polysaccharide chain, which forms the hydrophilic (water-attracting) part of the surfactant. Useful polyglucoside surfactants include those having the following formula (XV):

R¹—O—(R²O)ₙ—Z(x) (XV)
wherein R₁ is an alkyl group having 8-18 carbon atoms;
R₂ is an ethylene or propylene group;
Z is a saccharide group with 5 to 6 carbon atoms;
n is an integer from 0 to 10; and
x is an integer from 1 to 5.

Nonlimiting examples of polyglucoside surfactants include decyl glucoside, lauryl glucoside, coco-glucoside, caprylyl/capryl glucoside, hexyl glucoside, octyl glucoside, decyl glucoside/coco-glucoside blend, lauryl glucoside/coco-glucoside blend, coco glucoside/benzyl alcohol, coco-glucoside/lauryl glucoside blend, lauryl glucoside/caprylyl/capryl glucoside blend, decyl glucoside/caprylyl/capryl glucoside blend, alkyl glucosides (e.g., decyl glucoside and octyl glucoside), and sugar-based glucosides such as maltodextrin-glucoside.

The cleansing composition optionally includes one or more polyglucoside surfactants. Nonetheless, in a preferred embodiment, the cleansing composition is free or essentially free from polyglucoside surfactants.

(c)(iii) Additional Nonionic Surfactants

Nonionic surfactants also include, for example, alcohols, alpha-diols, alkylphenols and esters of fatty acids, optionally being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; and combinations thereof.

Nonionic surfactants may be polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and combinations thereof.

Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and combinations thereof.

As glyceryl esters of fatty acids, glyceryl stearate (glyceryl mono-, di- and/or tristearate) (CTFA name: glyceryl stearate) or glyceryl ricinoleate and combinations thereof can in particular be used.

As glyceryl esters of C₈-C₂₄ alkoxylated fatty acids, polyethoxylated glyceryl stearate (glyceryl mono-, di- and/or tristearate) such as PEG-20 glyceryl stearate can for example be used.

Mixtures of these surfactants, such as for example the product containing glyceryl stearate and PEG-100 stearate, marketed under the name “ARLACEL 165” by Uniqema, and the product containing glyceryl stearate (glyceryl mono- and distearate) and potassium stearate marketed under the name “TEGIN” by Goldschmidt (CTFA name: glyceryl stearate SE), can also be used.

The gentle cleansing composition includes one or more nonionic surfactants, preferably, one or more alkoxylated nonionic surfactants. Alkoxylated nonionic surfactants are a class of surfactants formed by the reaction of hydrophilic molecules, such as alcohols, phenols, or fatty acids, with alkylene oxides like ethylene oxide (EO) or propylene oxide (PO). Nonlimiting examples include alkoxylated polysorbates, alkoxylated sorbitan esters, alkoxylated fatty ethers, alkoxylated fatty acids, alkoxylated glyceryl esters, or combinations thereof. In various embodiments, the alkoxylated nonionic surfactants are selected from ethoxylated fatty alcohols, polysorbates, PEG derivatives, and poloxamers.

Nonlimiting examples of ethoxylated fatty alcohols include, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are mentioned. In at least one instance, the one or more alkoxylated fatty alcohols include steareth-20. In some instances, the one or more alkoxylated fatty alcohols may be exclusively steareth-20.

Polysorbates are nonionic surfactants made by the ethoxylation of sorbitan (a derivative of sorbitol) combined with fatty acids. Nonlimiting examples include polyoxyethylenated C8-C30 fatty acid esters (preferably C12-C18) of sorbitan, in particular lauric, myristic, cetylic or stearic acids, of sorbitan, especially containing from 2 to 30 mol of ethylene oxide, such as: polyoxyethylenated sorbitan monolaurate (4 EO) (Polysorbate-21) (HLB 13.3) polyoxyethylenated sorbitan monolaurate (20 EO) (Polysorbate-20) (HLB 16.7) polyoxyethylenated sorbitan monopalmitate (20 EO) (Polysorbate-40) (HLB 15.6) polyoxyethylenated sorbitan monostearate (20 EO) (Polysorbate-60) (HLB 14.9) polyoxyethylenated sorbitan monostearate (4 EO) (Polysorbate-61) (HLB 9.6) polyoxyethylenated sorbitan monooleate (20 EO) (Polysorbate-80) (HLB 15).

Nonlimiting examples of PEG derivatives include ethoxylated and hydrogenated castor oil, for example, PEG-7 hydrogenated castor oil, PEG-12 hydrogenated castor oil, PEG-20 hydrogenated castor oil, PEG-40 hydrogenated castor oil, PEG-60 hydrogenated castor oil, PEG-80 hydrogenated castor oil, and rapeseedamide ethoxylates, for example, PEG-2 rapeseedamide, PEG-4 rapeseedamide, PEG-6 rapeseedamide, PEG-10 rapeseedamide, PEG-20 rapeseedamide, PEG-30 rapeseedamide, PEG-40 rapeseedamide, PEG-60 rapeseedamide, PEG-80 rapeseedamide. Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and combinations thereof.

Poloxamers are a class of block copolymers made by combining ethylene oxide (EO) and propylene oxide (PO) units. Nonlimiting examples of poloxamers include poloxamer 188, poloxamer 407, poloxamer 338, poloxamer 237, poloxamer 908, poloxamer 124, poloxamer 181, poloxamer 184, poloxamer 331, poloxamer 105, poloxamer 103, poloxamer 188, and poloxamer 205.

In various embodiments, the one or more alkoxylated nonionic surfactants has a Hydrophobic-Lipophilic Balance (HLB) of 10 or higher. Such nonionic surfactants may be selected from alkoxylated polysorbates, alkoxylated sorbitan esters, alkoxylated fatty ethers, alkoxylated fatty acids, alkoxylated glyceryl esters, or combinations thereof. Nonlimiting examples of alkoxylated nonionic surfactants having an HLB of 10 or higher include PEG-20 stearate, PEG-40 stearate, PEG-100 stearate, PEG-20 laurate, PEG-8 laurate, PEG-40 laurate, PEG-150 distearate, PEG-7 cocoate, PEG-9 cocoate, PEG-8 oleate, PEG-10 oleate and PEG-40 hydrogenated castor oil, polysorbate 85, polysorbate 60, polysorbate 80, cetearyl glucoside, oleth-10, ceteth-10, ceteareth-20, or a mixture thereof. In a preferred embodiment, the one or more alkoxylated nonionic surfactants having an HLB of 10 or higher are selected from PEG-20 stearate, PEG-40 stearate, PEG-100 stearate, PEG-40 laurate, PEG-20 laurate, PEG-150 distearate, polysorbate 85, polysorbate 60, polysorbate 80, cetearyl glucoside, oleth-10, ceteth-10, ceteareth-20, PEG-8 laurate, PEG-9 cocoate, PEG-7 cocoate, PEG-8 oleate, PEG-40 hydrogenated castor oil, PEG-12 dimethicone, PEG-75 lanolin, PEG-150 distearate, PEG-100 lanolate, PEG-60 almond glycerides, PEG-150 PPG-2 stearate, PEG-1000 stearate, PEG-20 cetyl ether, PEG-40 castor oil, PEG-200 stearate, PEG-150 cetyl ether, PEG-4000 stearate, PEG-4 dilaurate, PEG-25 laureth-4, PEG-20 hydrogenated lanolin, and PEG-14 caprylate.

The total amount of the one or more nonionic surfactants in the cleansing composition will vary. Nonetheless, the total amount of the one or more nonionic surfactants is greater than zero but typically less than 10 wt. %, based on the total weight of the gentle cleansing composition. In further embodiments, the cleansing composition includes an amount greater than zero but less than 8 wt. %, less than 6 wt. %, less than 5 wt. %, less than 4 wt. %, less than 3 wt. %, or less than 2 wt. %, based on the total weight of the gentle cleansing composition. In further embodiments, the cleansing composition includes from about 0.1 to about 10 wt. %, about 0.1 to about 8 wt. %, about 0.1 to about 5 wt. %, about 0.1 to about 3 wt. %, about 0.1 to about 2 wt. %, of one or more nonionic surfactants. Preferably, however, the gentle cleansing composition includes from about 0.5 to about 10 wt. % of one or more nonionic surfactants, based on the total weight of the gentle cleansing composition. In further preferred embodiments, the gentle cleansing composition includes form about 0.5 to about 8 wt. %, about 0.5 to about 6 wt. %, about 0.5 to about 5 wt. %, about 0.8 to about 10 wt. %, about 0.8 wt. % to about 8 wt. %, about 0.8 to about 6 wt. %, about 0.8 to about 5 wt. %, about 1 to about 10 wt. %, about 1 to about 8 wt. %, about 1 to about 6 wt. %, or about 1 to about 5 wt. % of the one or more nonionic surfactants, based on the total weight of the gentle cleansing composition.

Preferably, the gentle cleansing composition includes form about 0.5 to about 8 wt. %, more preferably about 0.8 to about 5 wt. %, even more preferably about 1 to about 4 wt. % of the one or more nonionic surfactants, based on the total weight of the gentle cleansing composition.

As noted above, it is beneficial if one or more of the nonionic surfactants is an alkoxlyated nonionic surfactant. The total amount of one or more alkoxylated nonionic surfactants will vary but is typically from about 0.1 to about 5 wt. %, based on the total weight of the gentle cleansing composition. In further embodiments, the gentle cleansing composition includes from about 0.1 to about 3 wt. %, about 0.1 to about 2 wt. %, about 0.1 to about 1 wt. %, about 0.5 to about 5 wt. %, about 0.5 to about 3 wt. %, about 0.5 to about 2 wt. %, about 0.5 to about 1 wt. %, about 1 to about 5 wt. %, about 1 to about 3 wt. %, or about 1 to about 2 wt. %, of one or more alkoxylated nonionic surfactants, based on the total weight of the gentle cleansing composition. Preferably, the gentle cleansing composition includes about 0.1 to about 5 wt. %, more preferably about 0.5 to about 4 wt. %, and even more preferably about 1 to about 3 wt. % of one or more alkoxylated nonionic surfactants, based on the total weight of the gentle cleansing composition.

### Combined Amounts of (a), (b), and (c)

The combined amount of the brassicyl valinate esylate (a), the one or more fatty alcohols (b), and the one or more nonionic surfactants (c), will vary but is typically from about 2 to about 12 wt. %, based on the total weight of the gentle cleansing composition. In further embodiments, the combined amount of (a), (b), and (c) in the hair treatment composition is from about 2 to about 10 wt. %, about 2 to about 8 wt. %, about 2 to about 6 wt. %, about 2 to about 5 wt. %, about 2.5 to about 12 wt. %, about 2.5 to about 10 wt. %, about 2.5 to about 8 wt. %, about 2.5 to about 6 wt. %, about 2.5 to about 5 wt. %, about 3 to about 12 wt. %, about 3 to about 10 wt. %, about 3 toa bout 8 wt. %, about 3 to about 6 wt. %, about 3 to about 5 wt. %, about 3.5 to about 12 wt. %, about 3.5 to about 10 wt. %, about 3.5 to about 8 wt. %, about 3.5 to about 6 wt. %, or about 3.5 to about 5 wt. %, based on the total weight of the gentle cleansing composition.

Preferably, the combined amount of (a), (b), and (c) is from about 2 to about 10 wt. %, more preferably about 2.5 to about 8 wt. %, even more preferably about 3 to about 6 wt. %, and even more preferably about 3.5 to about 5 wt. %, based on the total weight of the gentle cleansing composition.

### (d) Skin Active Agents

A skin active agent in the context of the present disclosure refers to an ingredient that provides a beneficial effect on the skin beyond basic cleansing. These agents are included, for example, to enhance skin hydration, exfoliation, repair, protection, or other dermatological benefits.

Nonlimiting examples of skin active agents include madecassoside, retinoic acid, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate;—derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech;—extracts of Pygeum afrianum such as that sold under the name Pygeum afrianum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Serobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of meadowsweet (Spiraea ulmaria) such as that sold under the name Cytobiol Ulmaire by societeLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaja saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C12-C13) sold under the name COSMACOL.RTM ECI by Sasol; trialkyl citrate (C14-C15) sold under the name COSMACOL.RTM. ECL by Sasol—10-hydroxydecanoic acid, including mixtures acid-hydroxydecanoic October 25, sebacic acid and 1,10-decandiol such as that sold under the name Acnacidol BG by Vincience and mixtures thereof.

Skin active agents include cinnamic acid and derivatives thereof. Cinnamic acid or a derivative thereof may be selected from ferulic acid, p-coumaric acid, caffeic acid, sinapinic acid, chlorogenic acids, caftaric acid, chicoric acid, coutaric acid, rosmarinic acid, derivatives thereof, and combinations thereof. Equivalent derivatives thereof include those cinnamic acid derivatives having substitutions on the hydroxyl groups of the aromatic ring such as short chain aliphatic groups (one to six carbon atoms) or long chain aliphatic groups (seven to twenty-four carbon atoms) to form an ether, or such aliphatic groups substituted with alkyl, alkoxy, hydroxyl, amino, or amido, for example, to form a substituted ether. Equivalent derivatives thereof further include those cinnamic acid derivatives having modifications of the methoxy group(s) of the aromatic ring to short chain aliphatic groups (two to six carbon atoms) or to long chain aliphatic groups (seven to twenty-four carbon atoms) to form a longer chain ether, or such aliphatic groups substituted with alkyl, alkoxy, hydroxyl, amino, or amido, for example, to form a substituted long chain ether. The 3-carboxy group of a cinnamic acid derivative may also be converted to esters or amides having aliphatic groups of up to 24 carbons or an aromatic group, for example. Cis and trans isomers of the cinnamic acid derivatives are included herein since the cis isomer is readily converted to the trans isomer. Salts of the cinnamic acid derivatives are included herein. In one embodiment, the cinnamic acid derivative is a triethanolamine salt. Caffeic acid, also known as 3-(3,4-dihydroxyphenyl)-2-propenoic acid, is found in many fruits, vegetables, seasonings and beverages consumed by humans. Caffeic acid is present in such goods in conjugated forms such as chlorogenic acid. Para-coumaric acid, also known as 3-(4-hydroxyphenyl)-2-propenoic acid or p-hydroxycinnamic acid, is found in various plants, including lignin forming plants. Trans-ferulic acid, also known as 3-(4-hydroxy-3-methoxyp-phenyl)-2-propenoic acid or 4-hydroxy-3-methoxycinnamic acid, is also widely distributed in small amounts in plants. Sinapinic acid, also known as 3,5-dimethoxy4-hydroxycinnamic acid, is from black mustard seeds. Caffeic acid, para-coumaric acid, trans-ferulic acid and sinapinic acid are commercially available.

Skin active agents include hydroxy acids selected from alpha, beta or polyhydroxy acids. Nonlimiting examples include lactic acid, glycolic acid, salicylic acid, malic acid, tartaric acid, citric acid, mandelic acid, lactobionic acid, gluconolactone, galactose, and combinations thereof.

In various embodiments, one or more of the following skin active agents are selected from sodium hyaluronate, capryloyl salicylic acid, coco-caprylate/caprate, alpha and/or beta arbutin, ferulic acid, lucinol, kojic acid, resorcinol, tranexamic acid, gentisic acid, homogentisic, methyl gentisate or homogentisate, dioic acid, D pantheteine calcium sulphonate, lipoic acid, ellagic acid, vitamin B3, linoleic acid, stilbenoid, coumarin, tannin, curcuminoid, chalcone, phenylpropanoid, anthocyanin, dihydrochalcone, anthocyanidin, tocopherols, sesame lignin, vitamin C, caffeine, vitamin E, derivatives thereof, salts thereof, and a mixture thereof. In a preferred embodiment, the one or more skin agent agents are selected from Vitamin C, Vitamin E, caffeine, niacinamide, or a combination thereof.

Various skin active ingredients may be categorized as antioxidants, depigmenting agents, and anti-wrinkle agents.

(d)(i) Antioxidants

Examples of antioxidants include tocopherols (e.g. d-α-tocopherol, d-β-tocopherol, d-γ-tocopherol, d-delta-tocopherol), tocotrienols (e.g. d-α-tocotrienol, d-β-tocotrienol, d-γ.-tocotrienol, d-delta-tocotrienol) and vitamin E (α-tocopherol acetate). These compounds may be isolated from natural sources, prepared by synthetic means, or mixtures thereof. Tocotrienol-enriched vitamin E preparations may be obtained by fractionating vitamin E preparations to remove a portion of tocopherols and recover a preparation more highly concentrated in tocotrienol. Useful tocotrienols are natural products isolated, for example, from wheat germ oil, grain, or palm oil using high performance liquid chromatography, or isolated by alcohol extraction and/or molecular distillation from barley, brewer's grain or oats. As used herein, the term “tocotrienols” includes tocotrienol-rich-fractions obtained from these natural products as well as the pure compounds. The increased glutathione peroxidase activity protects the skin from oxidative damage.

Vitamin C and derivatives may be used, including ascorbic acid, sodium ascorbate, and the fat soluble esters tetrahexyldecyl ascorbate and ascorbyl palmitate, magnesium ascorbyl phosphate, ascorbyl-glucoside, glucosamine ascorbate, ascorbyl acetate, etc. Additionally, extracts from plants containing a high amount of vitamin C such as camu berry (Myrciaria dubia), acerola, emblica officinalis, and bioflavonoids from rose hip and citrus may be used including water soluble bioflavonoids such as hesperidin methyl chalcone may also be used.

Sesame (Sesamum indicum) or sesame lignan may also be added. Sesame and its lignans (the fibrous compounds associated with the sesame) act as antioxidants. Sesame seed lignans significantly enhance vitamin E activity.

In addition, carotenoids, particularly the xanthophyll type, are also useful antioxidants that can be used. The xanthopyll type carotenoids include molecules, such as lutein, canthaxantin, cryptoxanthin, zeaxanthin and astaxanthin. Xanthophylls protect compounds, such as vitamin A, vitamin E, and other carotenoids.

Flavonoids can also function as antioxidants. In some instances, the flavonoid is a flavanone (derivative of 2,3-dihydro-2-phenylchromen-4-one). Flavones include: Butin, Eriodictyol, Hesperetin, Hesperidin, Homoeriodictyol, Isosakuranetin, Naringenin, Naringin, Pinocembrin, Poncirin, Sakuranetin, Sakuranin, and Sterubin. The flavonoid may be a flavanonol (derivative of 3-hydroxy-2,3-dihydro-2-phenylchromen-4-one). Flavanols include: Taxifolin, Aromadedrin, Chrysandroside A, Chrysandroside B, Xeractinol, Astilbin, and Fustin. The flavonoid may be a flavone (derivative of 2-phenylchromen-4-one). Flavones include: Apigenin, Luteolin, Tangeritin, Chrysin, Baicalein, Scutellarein, Wogonin, Synthetic Flavones: Diosmin, and Flavoxate. The flavonoid may be a flavonol (derivative of 3-hydroxy-2-phenylchromen-4-one). Flavonols include: 3-Hydroxyflavone, Azaleatin, Fisetin, Galangin, Gossypetin, Kaempferide, Kaempferol, Isorhamnetin, Morin, Myricetin, Natsudaidain, Pachypodol, Quercetin, Rhamnazin, Rhamnetin, Azalein, Hyperoside, Isoquercitin, Kaempferitrin, Myricitrin, Quercitrin, Robinin, Rutin, Spiraeoside, Xanthorhamnin, Amurensin, Icariin, and Troxerutin. The flavonoid may be a flavan-3-ol (derivatives of 2-phenyl-3,4-dihydro-2H-chromen-3-ol). Flavan-3-ols include: Catechin, Epicatechin, Epigallocatechin, Epicatechin gallate, Epigallocatechin gallate, Epiafzelechin, Fisetinidol, Guibourtinidol, Mesquitol, and Robinetinidol. The flavonoid may be a flavan-4-ol (derivative of 2-phenylchroman-4-ol). Flavan-4-ols include: Apiforol and Luteoforol. The flavonoid may be an isoflavone (derivative of 3-phenylchromen-4-one). Isoflavones include: Genistein, Daidzein, Biochanin A, Formononetin, and the Equol metabolite from Daidzein.

The antioxidant may be an anthocyanidin (derivative of 2-phenylchromenylium cation). Anthocyanidins include: Aurantinidin, Cyanidin, Delphinidin, Europinidin, Luteolinidin, Pelargonidin, Malvidin, Peonidin, Petunidin, Rosinidin, and Xanthone.

The antioxidant may be a Dihydrochalcone (derivative of 1,3-diphenyl-1-propanone). Dihydrochalcones include: Phloretin, Dihydrochalcone phloretin Phlorizin, Aspalathin, Naringin dihydrochalcone, Neohesperidin dihydrochalcone, and Nothofagin. Without limiting the mode of action of the invention, dihydrochalcones may exert an antioxidant effect by reducing reactive free radicals, like reactive oxygen and reactive nitrogen species.

The antioxidant may be an anthocyanin. Anthocyanins and their derivatives are antioxidants. Anthocyanins encompasses a class of flavonoid compounds that are naturally occurring, water-soluble compounds, responsible for the red, purple, and blue colors of many fruits, vegetables, cereal grains, and flowers. Additionally, anthocyanins are collagenase inhibitors. The inhibition of collagenase helps in the prevention and reduction of wrinkles, increase in skin elasticity, etc., which are caused by a reduction in skin collagen. The anthocyanins may be obtained from any portion of various plant sources, such as the fruit, flower, stem, leaves, root, bark, or seeds. One of skill in the art will understand that certain portions of the plant may contain higher natural levels of anthocyanins, and, therefore, those portions are used to obtain the desired anthocyanins. In some instances, antioxidants may include one or more betacyanin. Betacyanins, like anthocyanins, may be obtained from natural sources and are antioxidants.

The antioxidant may be a Phenylpropanoid (derivatives of cinnamic acid). Phenylpropanoids include: Cinnamic acid, Caffeic acid, Ferulic acid, Trans-ferulic acid (including its antioxidant pharmacore 2,6-dihydroxyacetophenome), 5-Hydroxyferulic acid, Sinapic acid, Coumaryl alcohol, Coniferyl alcohol, Sinapyl alcohol, Eugenol, Chavicol, Safrole, P-coumaric acid, and Sinapinic acid. Without limiting the mode of action of the invention, Phenylpropanoids may neutralize free radicals.

The antioxidant may be a Chalcone (derivative of 1,3-diphenyl-2-propen-1-one). Chalcones include: Butein, Okanin, Carthamin, Marein, Sophoradin, Xanthohumol, Flavokvain A, Flavokavain B, Flavokavin C, and synthetic Safalcone.

The antioxidant may be a Curcuminoid. Curcuminoids include: Curcumin, Desmethoxycurcumin, bis-Desmethoxycurcumin, Tetrahydrocurcumin, and Tetrahydrocurcuminoids. Curcumin and tetrahydrocurcuminoids may be derived from rhizomes of Curcuma longa. Tetrahydrocurcumin, a metabolite of curcumin, has been found to be a more potent antioxidant and more stable compared to curcumin.

The antioxidant may be a Tannin. Tannins include: Tannin, Terflavin B, Glucogallin, Dgallic acid, and Quercitannic acid.

The antioxidant may be a stilbenoid. Stilbenoids include: Resveratrol, Pterostilbene, and Piceatannol. Resveratrol may include, but is not limited to, 3,5,4′-trihydroxystilbene, 3,4,3′,5′-tetrahydroxystilbene (piceatannol), 2,3′,4,5′-tetrahydroxystilbene (oxyresveratrol), 4,4′-dihydroxystilbene, and alpha and beta glucoside, galactoside and mannoside derivatives thereof.

The antioxidant may be a Coumarin (derivatives of 2H-chromen-2-one). Coumarins include: 4-Hydroxycoumarin, Umbelliferone, Aesculetin, Herniarin, Auraptene, and Dicoumarol.

The antioxidant may be a Carotenoid. Carotenoids include: beta-Carotene, alpha-Carotene, gamma-Carotene, beta-Cryptoxanthin, Lycopene, Lutein, and Idebenone. Sesame (Sesamum indicum) or sesame lignan may also be added. Sesame and its lignans (the fibrous compounds associated with the sesame) act as antioxidants. Sesame seed lignans significantly enhance vitamin E activity.

The antioxidant may be: a Xanthone, Butylated Hydroxytoluene, 2,6-Di-tert-butylphenol, 2,4-Dimethyl-6-tert-butylphenol, Gallic acid, Eugenol, Uric acid, alpha-Lipoic acid, Ellagic acid, Chicoric acid, Chlorogenic acid, Rosmarinic acid, Salicylic acid, Acetylcysteine, S-Allyl cysteine, Barbigerone, Chebulagic acid, Edaravone, Ethoxyquin, Glutathione, Hydroxytyrosol, Idebenone, Melatonin, N-Acetylserotonin, Nordihydroguaiaretic acid, Oleocanthal, Oleuropein, Paradol, Piceatannol, Probucol, Propyl gallate, Protocatechuic acid, Pyritinol, Rutin, Secoisolariciresinol diglucoside, Sesamin, Sesamol, Silibinin, Silymarin, Theaflavin, Theaflavin digallate, Thmoquinone, Trolox, Tyrosol, Polyunsaturated fatty acids, and sulfur-based antioxidants such as Methionine or Lipoic acid.

(d)(ii) Depigmenting Agents

Nonlimiting examples of depigmenting agents include alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, certain compounds derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

(d)(iii) Anti-Wrinkle Agent

The term “anti-wrinkle agent” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Nonlimiting examples of anti-wrinkle agents include: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof. Examples of such compounds are: adenosine and its derivatives and retinoids other than retinol (as discussed above, such as retinol palmitate), ascorbic acid and its derivatives such as magnesium ascorbyl phosphate and ascorbyl glucoside; nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the α-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof. In at least one case, the skin perfecting and tightening composition includes adenosine derivatives, such as non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside. Other derivatives include adenosine receptor agonists such as adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′-N-ethylcarboxamido adenosine (CGS-21680), N-ethylcarboxamido-adenosine (NECA), the 5′ (N-cyclopropyl)-carboxamidoadenosine, DPMA (PD 129.944) and metrifudil.

The total amount of the one or more skin active agents in the skin perfecting and tightening compositions if present will vary. Nonetheless, in various embodiments, the skin perfecting and tightening compositions includes an amount greater than zero to about 10 wt. %, based on a total weight of the skin perfecting and tightening composition. In further embodiments, the skin perfecting and tightening composition includes an amount of skin active agents in an amount greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the skin perfecting and tightening composition.

(d)(iv) Alpha Hydroxy Acids

The total amount of the one or more skin active agents in the skin perfecting and tightening compositions if present will vary. Nonetheless, in various embodiments, the skin perfecting and tightening compositions includes an amount greater than zero to about 10 wt. %, based on a total weight of the skin perfecting and tightening composition. In further embodiments, the skin perfecting and tightening composition includes an amount of skin active agents in an amount greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the skin perfecting and tightening composition.

### (e) Water Soluble Solvents

The term “water soluble solvent” is interchangeable with the terms “water soluble organic solvent” and “water-miscible solvent” and means a compound that is liquid at 25 °C and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, C₂-C₆ monoalcohols, glycerin, polyols, glycols, glycol ethers, and mixtures thereof.

Nonlimiting examples of C₂-C₆ monoalcohols and polyols include ethanol, isopropyl alcohol, propyl alcohol, 1-butanol, 2-butanol, 2-methylpropan-1-ol, 2-methylpropanol, and mixtures thereof.

Water soluble solvents include glycols and glycol ethers such as, for example, monomethyl, monoethyl and monobutyl ethers of ethylene glycol, propylene glycol, such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Water soluble solvents include polyols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, and a mixture thereof.

Water soluble solvents are not necessarily required in the hair treatment compositions of the present disclosure and therefore, is some cases, the hair treatment composition may be free or essentially free from water soluble solvents. Nonetheless, in certain embodiments, use of one or more water soluble solvents is preferred. For example, in a preferred embodiment, the hair treatment compositions include one or more polyols selected from glycerin, propylene glycol, butylene glycol, pentylene glycol, hexylene glycol, caprylyl glycol, dipropylene glycol, and mixtures thereof. In and even more preferably embodiment, the one or more water soluble solvents comprise or consist of glycerin.

In various embodiments, the hair treatment composition is preferably free or essentially free from ethanol. In further preferred embodiments, the hair treatment composition is free or essentially free from ethanol and isopropanol. In an even more preferred embodiment, the hair treatment composition is free or essentially free from C₂-C₆ monoalcohols.

The total amount of the one or more water soluble solvents in the gentle cleansing compositions will vary but is typically from about 1 to about 20 wt. %, based on the total weight of the gentle cleansing composition. In further embodiments, the gentle cleansing compositions includes about 1 to about 18 wt. %, about 1 to about 15 wt. %, about 1 to about 12 wt. %, about 1 to about 10 wt. %, about 1 to about 8 wt. %, about 2 to about 20 wt. %, about 2 to about 18 wt. %, about 2 to about 15 wt. %, about 2 to about 12 wt. %, about 2 to about 10 wt. %, about 2 to about 8 wt. %, about 4 to about 20 wt. %, about 4 to about 18 wt. %, about 4 to about 15 wt. %, about 4 to about 12 wt. %, about 4 to about 10 wt. %, about 4 to about 8 wt. %, about 5 to about 20 wt. %, about 5 to about 18 wt. %, about 5 to about 15 wt. %, about 5 to about 12 wt. %, about 5 to about 10 wt. %, about 5 to about 8 wt. %, about 6 to about 20 wt. %, about 6 to about 18 wt. %, about 6 to about 15 wt. %, about 6 to about 12 wt. %, about 6 to about 10 wt. %, or about 6 to about 8 wt. % of the one or more water soluble solvents, based on the total weight of the gentle cleansing composition.

Preferably, the gentle cleansing composition includes form about 2 to about 18 wt. %, more preferably about 4 to about 15 wt. %, even more preferably about 5 to about 12 wt. %, and even more preferably about 6 to about 10 wt. % of one or more water soluble solvents, based on the total weight of the gentle cleansing composition.

### (f) Water

The total amount of water in the gentle cleansing compositions will vary but is typically about 60 to about 92 wt. %, based on the total weight of the gentle cleansing composition. In further embodiments, the gentle cleansing composition includes about 60 to about 90 wt. %, about 60 to about 88 wt. %, about 65 to about 92 wt. %, about 65 to about 90 wt. %, about 65 to about 88 wt. %, about 70 to about 92 wt. %, about 70 to about 90 wt. %, about 70 to about 88 wt. %, about 75 to about 92 wt. %, about 75 to about 90 wt. %, about 75 to about 88 wt. %, about 80 to about 92 wt. %, about 80 to about 90 wt. %, about 80 to about 88 wt. %, about 82 to about 92 wt. %, about 82 to about 90 wt. %, about 80 to about 88 wt. % of water, based on the total weight of the gentle cleansing composition.

Preferably, the gentle cleansing composition includes about 70 to about 92 wt. %, more preferably about 75 to about 90 wt. %, and even more preferably about 80 to about 88 wt. % of water, based on the total weight of the gentle cleansing composition.

### (g) Thickening Polymers

Thickening polymers for use in cleansing compositions are substances that are added to formulations to increase the viscosity (thickness) of the product, improving its texture and stability. These polymers help create a rich, smooth consistency that enhances the sensory experience during application, making the product easier to spread and less likely to drip. Thickening polymers are also used in aqueous cleansing products to manipulate the viscosity, usually to increase the viscosity to make them easier for the user to handle and/or to increase the yield stress of the composition. While a certain thickening polymers may thicken or enhance the viscosity of a composition in which it is included, it may not necessarily have desirable yield stress properties. A desirable yield stress property is critical to achieving certain physical and aesthetic characteristics in a liquid medium, such as overall formula stability. An acceptable yield stress value can impart a formula with adequate shelf life stability. It can also help suspend particles, insoluble liquid droplets, or gas bubbles within a liquid medium. Particles dispersed in a liquid medium will remain suspended if the yield stress (yield value) of the medium is sufficient to overcome the effect of gravity or buoyancy on those particles. Insoluble liquid droplets can be prevented from rising and coalescing and gas bubbles can be suspended and uniformly distributed in a liquid medium using yield value as a formulating tool. A yield stress fluid is used generally to adjust or modify the rheological properties of aqueous compositions. Such properties include, without limitation, viscosity improvement, flow rate improvement, stability to viscosity change over time, and the ability to suspend particles for indefinite periods of time.

Many thickening polymers are water-soluble, and increase the viscosity of water or form an aqueous gel when the cosmetic composition of the invention is dispersed/dissolved in water. The aqueous solution may be heated and cooled, or neutralized, for forming the gel, if necessary. The thickener may be dispersed/dissolved in an aqueous solvent that is soluble in water, e.g., ethyl alcohol when it is dispersed/dissolved in water. Non-imiting examples of various types of polymer thickeners include:

(g)(i) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. The carbomers are available as the Carbopol™ 900 series from B.F. Goodrich (e.g., Carbopol® 954). In addition, other suitable carboxylic acid polymeric agents include Carbopol Ultrez® 20 (Lubrizol Corp.), which is acrylates/C₁₀-C₃₀ alkyl acrylate crosspolymer, Ultrez® 10 (B.F. Goodrich), and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as Carbopol.RTM. 1342, Carbopol® 1382, Pemulen TR-1, and Pemulen TR-2, from B.F. Goodrich. In other words, examples of carboxylic acid polymer thickeners useful herein are those selected from carbomers, acrylates/C10-C30 alkyl acrylate crosspolymers, and mixtures thereof.

(g)(ii) Crosslinked Polyacrylate Polymers

The compositions of the present disclosure can optionally contain crosslinked polyacrylate polymers useful as thickeners or gelling agents including both cationic and nonionic polymers. Examples of useful crosslinked nonionic polyacrylate polymers and crosslinked cationic polyacrylate polymers are those described in U.S. Pat. Nos. 5,100,660, 4,849,484, 4,835,206, 4,628,078 U.S. Pat. No. 4,599,379 and EP 228,868, which are all incorporated herein by reference in their entirety.

(g)(iii) Polyacrylamide Polymers

The compositions of the present disclosure can optionally contain polyacrylamide polymers, especially nonionic polyacrylamide polymers including substituted branched or unbranched polymers. Among these polyacrylamide polymers is the nonionic polymer given the CTFA designation polyacrylamide and isoparaffin and laureth-7, available under the Tradename Sepigel 305 from Seppic Corporation. An exemplary polyacrylamide polymers is, for example, ammonium polyacryloyldimethyl taurate (Clariant Hostacerin AMPS).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc.

The compositions may also contain thickening and texturising gels of the type as exemplified by the product range called Lubrajel® from United Guardian. These gels have moisturizing, viscosifying, stabilizing properties.

(g)(iv) Gums and Polysaccharides

A wide variety of gums and polysaccharides can be useful herein as gelling agents. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation. Further polysaccharides include starch derivatives (for example, starch oxide, dialdehyde starch, dextrin, British gum, acetyl starch, starch phosphate, carboxymethyl starch, hydroxyethyl starch, hydroxypropyl starch).

Other useful polysaccharides include scleroglucans comprising a linear chain of (1-3) linked glucose units with a (1-6) linked glucose every three units, a commercially available example of which is Clearogel™. CS11 from Michel Mercier Products Inc.

Other thickening and gelling agents useful herein include materials which are primarily derived from natural sources. Nonlimiting examples of these gelling agents are gums such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

The total amount of the one or more thickening polymers, if present, will vary but is typically greater than zero to about 5 wt. %, based on the total weight of the gentle cleansing composition. In further embodiments, the gentle cleansing composition includes an amount greater than zero to about 4 wt. %, 3 wt. %, 2 wt. %, 1 wt. %, or 0.5 wt. %. For example, the gentle cleansing composition may includes from about 0.01 to about 5 wt. %, about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.3 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, or about 0.1 to about 0.5 wt. % of one or more thickening polymers, based on the total weight of the gentle cleansing composition.

In a preferred embodiment, the gentle cleansing composition includes about 0.01 to about 4 wt. %, more preferably about 0.05 to about 3 wt. %, and even more preferably about 0.1 to about 1 wt. % of one or more thickening polymers, based on the total weight of the gentle cleansing composition.

### (h) Miscellaneous Ingredients

The gentle cleansing composition optionally includes one or more miscellaneous ingredients. Miscellaneous ingredients are ingredients that are compatible with the skin perfecting and tightening compositions and do not disrupt or materially affect the basic and novel properties of the skin perfecting and tightening compositions. Miscellaneous ingredients commonly used in cosmetics are known in the art. Non-limiting examples include preservatives, fragrances, pH adjusters, salts, buffers, flavonoids, vitamins, botanical extracts, UV filtering agents, proteins, protein hydrolysates and/or isolates, hydrotropes, pearlescent agents, fillers, colorants, mattifying agents, etc.

In addition to the above, components described as optional component throughout the disclosure may also be construed as miscellaneous ingredients if not expressly set forth as an independent component of the skin perfecting and tightening composition. Thus, the term “miscellaneous ingredient” is understood as a catch-all phrase representing additional components that may optionally be present in amounts designated for the miscellaneous ingredients.

Miscellaneous ingredients can be included in the cosmetic composition, for example, in an amount of about 0.01 to about 10 wt. %, based on the total weight of the cosmetic composition. The total amount of the one or more miscellaneous ingredients may be about 0.01 to about 8 wt. %, about 0.01 to about 5 wt. %, about 0.01 to about 3 wt. %, about 0.1 to about 10 wt. %, about 0.1 to about 8 wt. %, about 0.1 to about 5 wt. %, about 0.1 to about 3 wt. %, about 1 to about 10 wt. %, about 1 to about 8 wt. %, about 1 to about 5 wt. %, or about 1 to about 3 wt. %, based on the total weight of the skin perfecting and tightening composition.

### PREFERRED EMBODIMENTS

In a preferred embodiment, the gentle cleansing composition comprises, consists essentially of, or consists of:
(a) about 0.1 to about 5 wt. %, preferably about 0.2 to about 3 wt. %, more preferably about 0.3 to about 2 wt. % of brassicyl valinate esylate;
(b) about 0.5 to about 8 wt. %, preferably about 1 to about 6 wt. %, more preferably about 2 to about 5 wt. %, and even more preferably about 3 to about 4 wt. % of one or more fatty alcohols, wherein preferably, the one or more fatty alcohols are solid fatty alcohols, preferably solid alcohols selected from myristyl alcohol, cetyl alcohol, stearyl alcohol, behenyl alcohol and combinations thereof such as cetylstearyl and cetearyl alcohol;
(c) about 0.5 to about 5 wt. %, preferably about 1 to about 4 wt. %, and even more preferably about 1.1 to about 3 wt. % of one or more nonionic surfactants, wherein preferably, at least one of the one or more nonionic surfactants is an alkoxylated nonionic surfactant;
(d) about 0.01 to about 5 wt. %, preferably about 0.1 to about 4 wt. %, and even more preferably about 0.2 to about 3 wt. % of one or more skin active agents, wherein preferably, the one or more skin active agents are selected from photoprotective agents, desquamating agents, depigmenting agents, moisturizing agents, skin lightening agents, anti-aging ingredients, anti-wrinkle agents, antioxidants, and vitamins, and even more preferably, the one or more skin active agents is a combination of skin active agents comprising:
   (i) lauroyl lactylate and/or a thereof;
   (ii) hyaluronic acid and/or a salt thereof;
   (iii) one or more ceramides;
   (iv) one or more alpha-hydroxy acids; and
   (v) optionally, cholesterol;
(e) about 1 to about 15 wt. %, preferably about 2 to about 12 wt. %, and more preferably about 5 to about 10 wt. % of one or more water soluble solvents, wherein preferably, the one or more water soluble solvents are selected from polyols with 2 to 8 carbon atoms, and even more preferably, the one or more water soluble solvents are selected from glycerin, ethylene glycol, propylene glycol, butylene glycol, pentylene glycol, hexylene glycol, caprylyl glycol, dipropylene glycol, diglycerin, polyglycerin-3, polyglycerin-6, polyglycerin-10, neopentyl glycol, trimethylolpropane, 1,2-octanediol; and
(f) about 60 to about 92 wt. %, preferably about 70 to about 91 wt. %, and more preferably, about 78 to about 90 wt. % of water;
(g) optionally, about 0.05 to about 5 wt. %, preferably about 0.08 to about 4 wt. %, more preferably about 0.1 to about 3 wt. % of one or more thickening polymers, wherein the one or more thickening polymers is preferably selected from polyacrylate polymers, polyacrylamide polymers, polysaccharides, and gums; and
(h) optionally, about 0.1 to about 10 wt. %, preferably about 0.5 to about 8 wt. %, and more preferably about 1 to about 5 wt. % of one or more miscellaneous ingredients;
   wherein the composition contains multilamellar vesicles, wherein the brassicyl valinate esylate is entrapped within the vesicles, and
   all weight percentages are based on a total weight of the composition.

Preferably, the gentle cleansing composition is free or essentially free from anionic surfactants.

Preferably, the gentle cleansing composition is free from quaternary amine surfactants.

Preferably, the gentle cleansing composition as a viscosity of about 5,000 to about 180,000 cps at 25 °C on a Brookfield viscometer using an RV spindle #6 (RVDV2+, spindle 6, 20 rpm).

Preferably, the gentle cleansing composition of claim 1, wherein the composition has a pH of about 4 to about 8, more preferably about 4 to about 7, even more preferably about 4 to less than 7, and even more preferably about 4 to about 6.

In a preferred embodiment, the gentle cleansing composition comprises (b) in an amount at least three times higher than (a).

### Combined Amounts of (a) and (b)

The combined amount of the brassicyl valinate esylate of (a) and the one or more nonionic surfactants of (b) in the gentle cleansing composition will vary, but is typically from about 1.5 to about 10 wt. %, based on the total weight of the hair treatment composition. In further embodiments, the combined amount of (a) and (b) in the hair treatment composition is from about 1.5 to about 8 wt. %, about 1.5 to about 6 wt. %, about 1.5 to about 5 wt. %, about 1.5 to about 4 wt. %, about 2 to about 8 wt. %, about 2 to about 6 wt. %, about 2 to about 5 wt. %, about 2 to about 4 wt. %, about 2.5 to about 8 wt. %, about 2.5 to about 6 wt. %, about 2.5 to about 5 wt. %, about 2.5 to about 4 wt. %, about 3 toa bout 8 wt. %, about 3 to about 6 wt. %, about 3 to about 5 wt. %, or about 3 to about 4 wt. %, based on the total weight of the gentle cleansing composition.

Preferably, the combined amount of (a) and (b) is from about 1.5 to about 8 wt. %, more preferably about 2 to about 6 wt. %, even more preferably about 2.5 to about 5 wt. %, and even more preferably about 3 to about 4 wt. %, based on the total weight of the gentle cleansing composition.

The combined amount of the brassicyl valinate esylate of (a) and the one or more nonionic surfactants of (b) in the gentle cleansing composition will vary, but is typically from about 2 to about 12 wt. %, based on the total weight of the gentle composition. In further embodiments, the combined amount of (a) and (b) is from about 2 to about 10 wt. %, about 2 to about 8 wt. %, about 2 to about 6 wt. %, about 2 to about 5 wt. %, about 2 to about 4 wt. %, about 2.5 to about 12 wt. %, about 2.5 to about 10 wt. %, about 2.5 to about 8 wt. %, about 2.5 to about 6 wt. %, about 2.5 to about 5 wt. %, about 2.5 to about 4 wt. %, about 3 to about 12 wt. %, about 3 to about 10 wt. %, about 3 to about 8 wt. %, about 3 to about 6 wt. %, about 3 to about 5 wt. %, or about 5 to about 4 wt. %, based on the total weight of the gentle cleansing composition.

### Combined Amounts of (a), (b), and (c)

The combined amount of the brassicyl valinate esylate (a), the one or more fatty alcohols (b), and the one or more nonionic surfactants (c), will vary but is typically from about 2 to about 12 wt. %, based on the total weight of the gentle cleansing composition. In further embodiments, the combined amount of (a), (b), and (c) in the hair treatment composition is from about 2 to about 10 wt. %, about 2 to about 8 wt. %, about 2 to about 6 wt. %, about 2 to about 5 wt. %, about 2.5 to about 12 wt. %, about 2.5 to about 10 wt. %, about 2.5 to about 8 wt. %, about 2.5 to about 6 wt. %, about 2.5 to about 5 wt. %, about 3 to about 12 wt. %, about 3 to about 10 wt. %, about 3 toa bout 8 wt. %, about 3 to about 6 wt. %, about 3 to about 5 wt. %, about 3.5 to about 12 wt. %, about 3.5 to about 10 wt. %, about 3.5 to about 8 wt. %, about 3.5 to about 6 wt. %, or about 3.5 to about 5 wt. %, based on the total weight of the gentle cleansing composition.

Preferably, the combined amount of (a), (b), and (c) is from about 2 to about 10 wt. %, more preferably about 2.5 to about 8 wt. %, even more preferably about 3 to about 6 wt. %, and even more preferably about 3.5 to about 5 wt. %, based on the total weight of the gentle cleansing composition.

In a preferred embodiment, the gentle cleansing composition comprises, consists essentially of, or consists of:
(a) about 0.1 to about 5 wt. %, preferably about 0.2 to about 3 wt. %, more preferably about 0.3 to about 2 wt. % of brassicyl valinate esylate;
(b) about 0.5 to about 8 wt. %, preferably about 1 to about 6 wt. %, more preferably about 2 to about 5 wt. %, and even more preferably about 3 to about 4 wt. % of one or more fatty alcohols, wherein preferably, the one or more fatty alcohols are solid fatty alcohols, preferably solid alcohols selected from myristyl alcohol, cetyl alcohol, stearyl alcohol, behenyl alcohol and combinations thereof such as cetylstearyl and cetearyl alcohol;
(c) about 0.5 to about 5 wt. %, preferably about 1 to about 4 wt. %, and even more preferably about 1.1 to about 3 wt. % of one or more nonionic surfactants, wherein preferably, at least one of the one or more nonionic surfactants is an alkoxylated nonionic surfactant selected from alkoxylated fatty alcohols, alkoxylated polysorbates, alkoxylated esters of fatty acids, and alkoxylated fatty ethers and at least one of the one or more nonionic surfactants is a non-alkoxylated nonionic surfactant;
(d) about 0.01 to about 5 wt. %, preferably about 0.1 to about 4 wt. %, and even more preferably about 0.2 to about 3 wt. % of one or more skin active agents, wherein preferably, the one or more skin active agents are selected from photoprotective agents, desquamating agents, depigmenting agents, moisturizing agents, skin lightening agents, anti-aging ingredients, anti-wrinkle agents, antioxidants, and vitamins, and even more preferably, the one or more skin active agents is a combination of skin active agents comprising:
   (i) lauroyl lactylate and/or a thereof;
   (ii) hyaluronic acid and/or a salt thereof;
   (iii) one or more ceramides;
   (iv) one or more alpha-hydroxy acids; and
   (v) optionally, cholesterol;
(e) about 1 to about 15 wt. %, preferably about 2 to about 12 wt. %, and more preferably about 5 to about 10 wt. % of one or more water soluble solvents, wherein preferably, the one or more water soluble solvents are selected from polyols with 2 to 8 carbon atoms, and even more preferably, the one or more water soluble solvents are selected from glycerin, ethylene glycol, propylene glycol, butylene glycol, pentylene glycol, hexylene glycol, caprylyl glycol, dipropylene glycol, diglycerin, polyglycerin-3, polyglycerin-6, polyglycerin-10, neopentyl glycol, trimethylolpropane, 1,2-octanediol, more preferably wherein the one or more water soluble solvents comprises or consists of glycerin; and
(f) about 60 to about 92 wt. %, preferably about 70 to about 91 wt. %, and more preferably, about 78 to about 90 wt. % of water;
(g) optionally, about 0.05 to about 5 wt. %, preferably about 0.08 to about 4 wt. %, more preferably about 0.1 to about 3 wt. % of one or more thickening polymers, wherein the one or more thickening polymers is preferably selected from polyacrylate polymers, polyacrylamide polymers, polysaccharides, and gums; and
(h) optionally, about 0.1 to about 10 wt. %, preferably about 0.5 to about 8 wt. %, and more preferably about 1 to about 5 wt. % of one or more miscellaneous ingredients, wherein preferably, the one or more miscellaneous ingredients are selected from preservatives, fragrances, pH adjusters, salts, buffers, flavonoids, vitamins, botanical extracts, UV filtering agents, proteins, protein hydrolysates and/or isolates, hydrotropes, pearlescent agents, fillers, colorants, mattifying agents;
   wherein the composition contains multilamellar vesicles, wherein the brassicyl valinate esylate is entrapped within the vesicles, and
   all weight percentages are based on a total weight of the composition.

Preferably, the gentle cleansing composition is free or essentially free from anionic surfactants.

Preferably, the gentle cleansing composition is free from quaternary amine surfactants.

Preferably, the gentle cleansing composition as a viscosity of about 5,000 to about 180,000 cps at 25 °C on a Brookfield viscometer using an RV spindle #6 (RVDV2+, spindle 6, 20 rpm).

Preferably, the gentle cleansing composition of claim 1, wherein the composition has a pH of about 4 to about 8, more preferably about 4 to about 7, even more preferably about 4 to less than 7, and even more preferably about 4 to about 6.

In a preferred embodiment, the gentle cleansing composition comprises (b) in an amount at least three times higher than (a).

### Combined Amounts of (a) and (b)

The combined amount of the brassicyl valinate esylate of (a) and the one or more nonionic surfactants of (b) in the gentle cleansing composition will vary, but is typically from about 1.5 to about 10 wt. %, based on the total weight of the hair treatment composition. In further embodiments, the combined amount of (a) and (b) in the hair treatment composition is from about 1.5 to about 8 wt. %, about 1.5 to about 6 wt. %, about 1.5 to about 5 wt. %, about 1.5 to about 4 wt. %, about 2 to about 8 wt. %, about 2 to about 6 wt. %, about 2 to about 5 wt. %, about 2 to about 4 wt. %, about 2.5 to about 8 wt. %, about 2.5 to about 6 wt. %, about 2.5 to about 5 wt. %, about 2.5 to about 4 wt. %, about 3 toa bout 8 wt. %, about 3 to about 6 wt. %, about 3 to about 5 wt. %, or about 3 to about 4 wt. %, based on the total weight of the gentle cleansing composition.

Preferably, the combined amount of (a) and (b) is from about 1.5 to about 8 wt. %, more preferably about 2 to about 6 wt. %, even more preferably about 2.5 to about 5 wt. %, and even more preferably about 3 to about 4 wt. %, based on the total weight of the gentle cleansing composition.

The combined amount of the brassicyl valinate esylate of (a) and the one or more nonionic surfactants of (b) in the gentle cleansing composition will vary, but is typically from about 2 to about 12 wt. %, based on the total weight of the gentle composition. In further embodiments, the combined amount of (a) and (b) is from about 2 to about 10 wt. %, about 2 to about 8 wt. %, about 2 to about 6 wt. %, about 2 to about 5 wt. %, about 2 to about 4 wt. %, about 2.5 to about 12 wt. %, about 2.5 to about 10 wt. %, about 2.5 to about 8 wt. %, about 2.5 to about 6 wt. %, about 2.5 to about 5 wt. %, about 2.5 to about 4 wt. %, about 3 to about 12 wt. %, about 3 to about 10 wt. %, about 3 to about 8 wt. %, about 3 to about 6 wt. %, about 3 to about 5 wt. %, or about 5 to about 4 wt. %, based on the total weight of the gentle cleansing composition.

### Combined Amounts of (a), (b), and (c)

The combined amount of the brassicyl valinate esylate (a), the one or more fatty alcohols (b), and the one or more nonionic surfactants (c), will vary but is typically from about 2 to about 12 wt. %, based on the total weight of the gentle cleansing composition. In further embodiments, the combined amount of (a), (b), and (c) in the hair treatment composition is from about 2 to about 10 wt. %, about 2 to about 8 wt. %, about 2 to about 6 wt. %, about 2 to about 5 wt. %, about 2.5 to about 12 wt. %, about 2.5 to about 10 wt. %, about 2.5 to about 8 wt. %, about 2.5 to about 6 wt. %, about 2.5 to about 5 wt. %, about 3 to about 12 wt. %, about 3 to about 10 wt. %, about 3 toa bout 8 wt. %, about 3 to about 6 wt. %, about 3 to about 5 wt. %, about 3.5 to about 12 wt. %, about 3.5 to about 10 wt. %, about 3.5 to about 8 wt. %, about 3.5 to about 6 wt. %, or about 3.5 to about 5 wt. %, based on the total weight of the gentle cleansing composition.

Preferably, the combined amount of (a), (b), and (c) is from about 2 to about 10 wt. %, more preferably about 2.5 to about 8 wt. %, even more preferably about 3 to about 6 wt. %, and even more preferably about 3.5 to about 5 wt. %, based on the total weight of the gentle cleansing composition.

### EXAMPLES

Implementation of the present disclosure is provided by way of the following examples. The examples serve to illustrate the technology without being limiting in nature.

### Example 1

### (Gentle Cleansing Compositions)

| | | | A |
|---|---|---|---|
| | | | wt. % |
| (a) | Cationic Surfactant | BRASSICYL VALINATE | 0.5 |
| | | ESYLATE (BVE) | |
| (b) | Fatty Alcohols | CETYL ALCOHOL | 0.9 |
| | | STEARYL ALCOHOL | 0.6 |
| | | CETEARYL ALCOHOL | 1.2 |
| Combination of (a) + (b) | | | 3.2 |
| (c) | Alkoxylated Nonionic | POLYSORBATE 20 | 0.2 |
| | emulsifiers | PEG-40 STEARATE | 0.5 |
| | | GLYCERYL STEARATE | 0.4 |
| | Nonionic Emulsifier | GLYCERYL STEARATE | 0.4 |
| Combination of (a) + (b) + (c) | | | 4.7 |
| (d) | Skin Active Agents | SODIUM HYALURONATE | ~0.3 |
| | | SODIUM LAUROYL | |
| | | LACTYLATE, CERAMIDE | |
| | | NP, CERAMIDE AP, | |
| | | PHYTOSPHINGOSINE, | |
| | | CHOLESTEROL, & | |
| | | CERAMIDE EOP | |
| (e) | Water Soluble Solvent | GLYCERIN | 7.5 |
| (f) | Water | WATER | 86.7 |
| (g) | Nonionic Polymer | XANTHAN GUM | ≤0.1 |
| | Thickener | CARBOMER | |
| (h) | Miscellaneous Ingredients | LACTIC ACID | 0.1 |
| | (pH adjusters, preservatives, | PHENOXYETHANOL | 0.9 |
| | chelating agents) | DISODIUM EDTA | 0.1 |

| | |
|---|---|
| pH | 3.8 |

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular.

The expression “at least one” means one or more and thus includes individual components as well as mixtures/combinations.

All ranges and values disclosed herein are inclusive and combinable. For examples, any value or point described herein that falls within a range described herein can serve as a minimum or maximum value to derive a sub-range, etc.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A gentle cleansing composition comprising:
(a) about 0.1 to about 5 wt. % of brassicyl valinate esylate;
(b) about 1 to about 8 wt. % of one or more fatty alcohols;
(c) about 0.5 to about 5 wt. % of one or more nonionic surfactants;
(d) about 0.01 to about 5 wt. % of one or more skin active agents; and
(e) about 1 to about 15 wt. % of one or more water soluble solvents;
(f) water;
wherein the composition contains multilamellar vesicles, wherein the brassicyl valinate esylate is entrapped within the vesicles, and
all weight percentages are based on a total weight of the composition.

2. The gentle cleansing composition of claim 1, wherein the one or more fatty alcohols are solid fatty alcohols selected from myristyl alcohol, cetyl alcohol, stearyl alcohol, behenyl alcohol and combinations thereof such as cetylstearyl or cetearyl alcohol.

3. The gentle cleansing composition of claim 1, wherein at least one of the one or more nonionic surfactants are selected from alkoxylated nonionic surfactants.

4. The gentle cleansing composition of claim 1, wherein the alkoxylated nonionic surfactants are selected from alkoxylated fatty alcohols, alkoxylated polysorbates, alkoxylated esters of fatty acids, and alkoxylated fatty ethers.

5. The gentle cleansing composition of claim 1, wherein the one or more skin active agents are selected from photoprotective agents, desquamating agents, depigmenting agents, moisturizing agents, skin lightening agents, anti-aging ingredients, anti-wrinkle agents, antioxidants, and vitamins.

6. The gentle cleansing composition of claim 5, wherein the one or more skin active agents are a combination of skin active agents comprising:
(i) lauroyl lactylate and/or a thereof;
(ii) hyaluronic acid and/or a salt thereof;
(iii) one or more ceramides;
(iv) one or more alpha-hydroxy acids; and
(v) optionally, cholesterol.

7. The gentle cleansing composition of claim 1, wherein the one or more water soluble solvents are selected from polyols with 2 to 8 carbon atoms.

8. The gentle cleansing composition of claim 7, wherein the polyols are selected from glycerin, ethylene glycol, propylene glycol, butylene glycol, pentylene glycol, hexylene glycol, caprylyl glycol, dipropylene glycol, diglycerin, polyglycerin-3, polyglycerin-6, polyglycerin-10, neopentyl glycol, trimethylolpropane, 1,2-octanediol.

9. The gentle cleansing composition of claim 1, wherein the composition further comprises one or more thickening polymers.

10. The gentle cleansing composition of claim 9, wherein the one or more thickening polymers are selected from polyacrylate polymers, polyacrylamide polymers, polysaccharides, and gums.

11. The gentle cleansing composition of claim 1, wherein the water is in an amount of about 80 to about 92 wt. %.

12. The gentle cleansing composition of claim 1, wherein the composition is free or essentially free from anionic surfactants.

13. The gentle cleansing composition of claim 1, wherein the composition is free from quaternary amine surfactants.

14. The gentle cleansing composition of claim 1, wherein the composition has a viscosity of about 5,000 to about 180,000 cps at 25 °C on a Brookfield viscometer using an RV spindle #6 (RVDV2+, spindle 6, 20 rpm).

15. The gentle cleansing composition of claim 1, wherein the composition has a pH of about 3.5 to about 6.

16. The gentle cleansing composition of claim 1, wherein (b) is in an amount at least three times higher than (a).

17. A gentle cleansing composition comprising:
(a) about 0.1 to about 5 wt. % of brassicyl valinate esylate;
(b) about 1 to about 8 wt. % of one or more solid fatty alcohols;
(c) about 1 to about 5 wt. % of one or more alkoxylated nonionic surfactants selected from alkoxylated fatty alcohols, alkoxylated polysorbates, alkoxylated esters of fatty acids, and alkoxylated fatty ethers;
(d) about 0.01 to about 5 wt. % of one or more skin active agents;
(e) about 1 to about 15 wt. % of one or more water soluble solvents with 2 to 8 carbon atoms selected from glycerin, ethylene glycol, propylene glycol, butylene glycol, pentylene glycol, hexylene glycol, caprylyl glycol, dipropylene glycol, and diglycerin;
(f) water;
(g) one or more thickening polymers selected from polyacrylate polymers, polyacrylamide polymers, polysaccharides, and gums; and
(h) optionally, about 0.01 to about 8 wt. % of one or more miscellaneous ingredients;
wherein the composition contains multilamellar vesicles, wherein the brassicyl valinate esylate is entrapped within the vesicles, and
all weight percentages are based on a total weight of the composition.

18. The gentle cleansing composition of claim 17, wherein the one or more skin active agents are a combination of skin active agents comprising:
(i) lauroyl lactylate and/or a thereof;
(ii) hyaluronic acid and/or a salt thereof;
(iii) one or more ceramides;
(iv) one or more alpha-hydroxy acids; and
(v) optionally, cholesterol.

19. A method for cleansing skin comprising applying the gentle cleansing composition of claim 1 to the skin and rinsing it from the skin with water.

20. The method of claim 19, wherein the skin is skin of the face.
